(19) United States
(12) Patent Application Publication
(10) Pub. No.: US 2003/0144212 A1
(43) Pub. Date: Jul. 31, 2003

(54) USE OF SERINE PROTEASE INHIBITORS TO INHIBIT PATHOPHYSIOLOGY AND NEUROPATHOLOGY IN A HOST

(76) Inventors: KEITH B HOFFMAN, SAN DIEGO, CA (US); GARY LYNCH, IRVINE, CA (US)

Correspondence Address: BOZICEVIC, FIELD & FRANCIS LLP, 200 MIDDLEFIELD RD, SUITE 200, MENLO PARK, CA, 94025, US

(21) Appl. No.: 09/582,964

(22) PCT Filed: Jan. 8, 1999
(86) PCT No.: PCT/US99/00465

(51) Int. Cl. A61K 31/47; A61K 38/08

(52) U.S. Cl. 514/17; 514/311

(57) ABSTRACT

Methods are provided for inhibiting cell adhesion molecule cleavage in brain tissue of a host. In the subject methods, an effective amount of a protease inhibitor, particularly serine protease inhibitors, such as those that inhibit tPA and related proteases, is administered to the host. The subject methods find use in the treatment and prevention of pathophysiology and neuropathology in a host, such as the treatment of a variety of pathological conditions resulting from pathophysiology and/or excitotoxicity. Specific pathological conditions in which the subject methods find use include epilepsy (and related seizure states) and neuronal damage associated with excessive glutamate activity, e.g. resulting from an acute event such as hypoxia, head trauma or stroke.

This invention was made with United States Government support under Grant No. 49620-95-1-0304 awarded by AFOSR. The United States Government has certain rights in this invention.

### INTRODUCTION

1. Field of the Invention

The field of the invention is methods of treating seizures and related neurological disorders.

2. Background of the Invention

Synaptic plasticity is natural physiological process that is associated with memory and learning. See Wang et al., J. Clin. Neurophysiology (July 1997) 14: 264-293. It has been found that activity-dependent short- and long-term changes in the strength of synaptic transmission, such as long-term potentiation, are important for memory processes, and that such changes can result from synaptic plasticity. Thus, some synaptic plasticity is normal and does not lead to neuropathological conditions.

However, where the magnitude of synaptic plasticity deviates from that required for normal physiological purposes, such as memory and learning, neuropathological conditions or diseases can arise. For example, synaptic plasticity can lead to the consolidation of excessive long-term potentiation and a concomitant increase in neuronal excitability. Ben Ari & Represa, Trends in Neuroscience (August 1990) 13:312-318. Such changes can, in turn, render the host more susceptible to seizures.

One well known disease condition characterized by the occurrence of seizures is epilepsy. While the manifestations of the disease have been analyzed for centuries, the underlying biochemical mechanism is not well understood. As such, treatments for epilepsy have traditionally focused on the use of antiepileptic drugs which ameliorate the symptoms of the disease, but do not affect the underlying biochemical cause of the disease and symptoms associated therewith, i.e. do not affect synaptic plasticity. Antiepileptic drugs that find, or have found, use in the treatment and management of epilepsy include: carbamazepine; sodium valproate; dilantin; ethisyxunudem; benzodiasepines, such as clonazepam, clobazepam, diazepam and nitrazepam; barbituate drugs, such as methylphenobarbitone, primidone and phenobarbitone; and the like.

While the above list of drugs indicates a wide variety of choices for the treatment of diseases associated with synaptic plasticity, such as epilepsy, there continues to be an interest in the elucidation of the biochemical mechanism underlying synaptic plasticity and the identification of more direct methods of treating diseases arising from abnormal synaptic plasticity.

Relevant Literature

Wang et al., J. Clin. Neurophysiology (July 1997) 14: 264-293 provides a review of synaptic plasticity.

Other references of interest in the field of synaptic plasticity research, including the treatment of conditions related thereto, include: Tsirka et al., Nature (Sep. 28, 1995) 377: 340; Osterwalder et al., EMBO J. (Jun. 17, 1996) 15: 2944-2953; Kreuger et al., J. Neurosci. (Dec. 1, 1997)17:8984-8996; Sheppard et al., Biochim Biophys Acta (Jan. 8, 1991) 1076: 156-160; Lagunowich et al., Neurotoxicology (1994) 15: 123-132; Carroll et al., Development (November 1994) 120: 3173-3813; Koide et al., Neurochem Res. (December 1993) 18: 1259-1262; and Qian et al., Nature (Feb. 4, 1993) 361: 453-457.

### SUMMARY OF THE INVENTION

Methods for inhibiting the cleavage of cell adhesion molecules in the brain of a host are provided. In the subject methods, an effective amount of a protease inhibitor, particularly a serine protease inhibitor, such as a tPA inhibitor, is administered to the host. The subject methods find use in depressing the chemistries responsible for generating longlasting changes in synaptic transmission (i.e. pathophysiology) and neuropathology. As such, the subject methods find use in the treatment of a variety of pathological conditions resulting from pathophysiology and/or glutamate-driven excitotoxicity. Specific pathological conditions for which the subject methods find use include epilepsy (and related seizure states) and neuronal damage triggered by excessive glutamate receptor activity.

### BRIEF DESCRIPTION OF THE FIGURES

FIG. 1 is a quantitative assessment of a 75 kDa cell adhesion molecule (CAM) fragment (“CAM75”) in samples incubated with EGTA, EDTA, or no chelators (“buffer”). The graph demonstrates that CAM75 increases upon incubation of tissue homogenates from adult hippocampus in the presence of chelators, but decreases without them.

FIG. 2 is a graph demonstrating how the levels of CAM75 change in response to injection of adult rats with: 10 mg/kg kainate (KA); 10 mg/kg kainate and 60 mg/kg ketamine, an NMDA receptor antagonist (KA/KETA); and saline. The graph demonstrates that kainate-induced seizures increase the concentration of CAM75 by activating NMDA receptors.

FIG. 3. provides a graph of percent changes in fluorescence when ultrasupernatants were incubated with 100 μM of the serine protease targets arginine-AMC (A-AMC) (open bars; n=4) or with the amino acid sequence found in the extracellular domains of cell adhesion molecules [arginine-serine-lysine-arginine] (ASLA-AMC) (solid bars, n=4) with or without the addition of AEBSF at 250 and 1000 μM. The results demonstrate that AEBSF inhibits cleavage of serine protease targets by endogenous protease(s) found in hippocampal ultrasupernatants. Data were quantified by normalization of complete inhibition of fluorescence to 100 percent.

FIG. 4 provides a graph of the results obtained when tPA (1.5 μg/ml) was incubated with 40 μM of the fluorescent substrates A-AMC (open bar; n=6) or ASLA-AMC (solid bar; n=6) and quantification involved normalization of the breakdown of the A-AMC substrate to 100 (arbitrary fluorescent units). The results indicate that tissue plasminogen activator (tPA) shows high specificity for proteolysis of the serine protease target sequence found in cell adhesion molecules.

FIG. 5A shows changes in slope of excitatory postsynaptic potentials (EPSPs), generated by stimulating axons with 0.1 msec electrical pulses in brain slices treated without (open circles; n=6) or with (solid circles; n=6) a 45 minute incubation of 250 μM AEBSF prior to theta burst stimulation (a stimulation pattern modeled after endogenous brain rhythms that consists of stimulating input fibers with short high-frequency bursts of electrical pulses: TBS); while FIG. 5B shows changes in slope of EPSPs in slices treated with 250 μM AEBSF 5 minutes (solid circles; n=5) or 30 minutes (open circles; n=6) after TBS. Traces are representative waveforms taken from the groups summarized in the graph. Calibration: 1 mV/10 sec. The results demonstrate that AEBSF blocks the consolidation of LTP if added before, but not after, theta burst stimulation (TBS); within slice comparisons.

FIG. 6 is graph of the average seconds of physiological “after-discharges” (i.e. seizure activity) in rats 10 days after an experimental procedure which consisted of repeated electrical stimulation of the hippocampus. Rats were injected 10 minutes before the stimulation trains with saline or AEBSF. *=paired two sided t-test; p=0.0075. (The difference between AEBSF test days was not significant; p=0.75).

FIG. 7 is a graph of the percent change in the duration of after-discharges per stimulation.

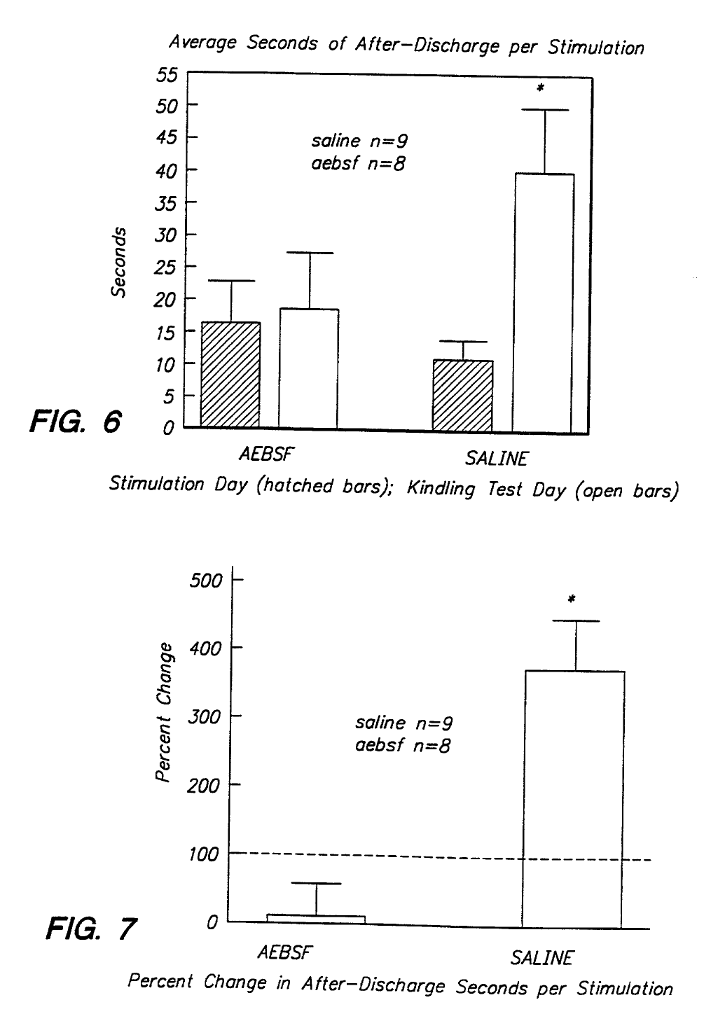

FIGS. 6 and 7 demonstrate that the normal reaction to multiple repetitive excitatory stimulations (which is a large increase in seizure-proneness) is blocked when serine proteases are inhibited during the stimulation procedures.

### DETAILED DESCRIPTION OF THE INVENTION

Methods are provided for inhibiting the cleavage of cell adhesion molecules in the brain of a host. In the subject methods, an effective amount of a protease inhibitor is administered to the host. The subject methods find use in treating a host having a neuropathological condition associated with 1) pathophysiology, and 2) neural degeneration caused by excess glutamate-driven proteolysis. Specific pathological conditions in which the subject methods find use include epilepsy (and related seizure states) and neuronal damage associated with excessive glutamate receptor activity, such as that resulting from hypoxia, head trauma or stroke.

Before the subject invention is further described, it is to be understood that the invention is not limited to the particular embodiments of the invention described below, as variations of the particular embodiments may be made and still fall within the scope of the appended claims. It is also to be understood that the terminology employed is for the purpose of describing particular embodiments, and is not intended to be limiting. Instead, the scope of the present invention will be established by the appended claims.

In this specification and the appended claims, the singular forms “a,” “an,” and “the” include plural reference unless the context clearly dictates otherwise. Unless defined otherwise. all technical and scientific terms used herein have the same meaning as commonly understood to one of ordinary skill in the art to which this invention belongs.

In the subject methods, an effective amount of a serine protease inhibitor is used to inhibit pathophysiology and neuropathology in a host. By inhibit is meant at least a reduction in the amount of pathophysiology and/or neuronal compromise as compared to a control, where inhibit includes situations where pathophysiology and/or neuronal compromise is completely stopped.

The subject methods find use in the treatment of hosts suffering from pathological conditions resulting from pathophysiology. By pathophysiology it is meant an undesirable increase in transmission between brain cells resulting from a change, alteration or modulation in the synaptic organization and/or responsiveness of the host, e.g. a synaptic ultrastructural reorganization and/or increase in responsiveness, and/or direct neuronal demise caused by, for example, excessive proteolytic activity.

Pathological conditions treated according to the subject invention are those in which the underlying pathophysiology and/or neuropathology is due to the cleavage of cell adhesion molecules, where the cleavage is an extracellular cleavage event of a cell adhesion molecule (CAM) found on the surface of cells of brain tissue. The cleavage event underlying the pathophysiology and/or neuropathology results from protease recognition of a CAM consensus sequence, particularly a sequence found in CAM fibronectin repeat domains, and more particularly the sequence of residues A-S-L-A (and close relatives to this sequence such as those which share the X-S-(A or L)-A sequence which all form a serine protease target site). The CAMs that are cleaved to produce the underlying pathophysiology/neuropathology that gives rise to conditions treated by the subject invention are related to, e.g. analogues of, NCAM (i.e. neural cell adhesion molecule, D2-CAM, CD56, Leu-19, NKHl), where the CAM is not NCAM. As such, the CAM cleaved in conditions treated by the subject invention may be members of the Ig-CAM family [Edelman, G. M. 1987. CAMs and Igs: Cell adhesion and the evolutionary origins of immunity. Immunol. Rev. 100: 11-45; Jessell, T. M. 1988. Adhesion molecules and the hierarchy of neural development. Neuron 1: 3-13] such as Ng-CAM (i.e. neuronal glial cell adhesion molecule), Tag-1,G4, 8D9, L1/NILE, Nr-CAM/Bravo, and analogues or related family members thereof, where generally the CAMs are cleaved by a serine protease to produce polypeptides with molecular weights generally from about 60 to 80 kDa, and more generally from about 75 kDa to 80 kDa, e.g. CAM75, and the ˜80 kDa fragments of Ng-CAM and L1.

Conditions resulting from pathophysiology include those in which synaptic stimulation results in an undesirable increase in long-term potentiation, where the term long-term potentiation is used herein to refer to a long lasting increase in synaptic efficacy following an appropriate stimulus, such as tetanic stimulation. Conditions treatable by the subject methods include those associated with the occurrence of “sprouting,” i.e. the appearance of mossy fibers which make multiple ectopic asymmetrical synapses with granule cell and basilar dendrites as described in see Repressa & Ben-Ari, Epilepsy Research. (1992 Supp.) 7:261-269, as the subject methods can inhibit and/or prevent sprouting. Such conditions include those characterized by the occurrence of seizures, where such conditions include epilepsy. Of particular interest is the use of the subject invention to treat hosts suffering from Status epilepticus and related seizure states, where treatment includes both the management of the disease, e.g. control of seizure proneness, as well as reversal of the progression of the disease, e.g. a decrease in seizure proneness.

Other pathological conditions that may be treated according to the subject invention include those conditions resulting from the demise of neurons (i.e. “neuropathology”) resulting from the excessive activity of glutamate receptors, particularly NMDA-type glutamate receptors, which excessive activity is the result of an acute event, such as hypoxia (e.g. cerebral ischemia), head trauma (cerebral trauma) or stroke.

In practicing the subject methods, an effective amount of a protease inhibitor is administered to the host in which depression of pathophysiology and/or neuropathology is desired, e.g. a host prone to seizures, such as an epileptic host or a host having a condition characterized by stroke, head trauma, and/or hypoxia. Protease inhibitors that find use in the subject invention include naturally occurring inhibitors, e.g. neuroserpin (Osterwalder et al.,EMBO J. (June 1996) 15: 2944-2953) and PAI-1, as well as synthetic analogues and mimetics thereof, particularly small molecule synthetic mimetics thereof, which are capable of at least reducing the ability of the protease to cleave CAMs. Of particular interest are serine protease inhibitors, particularly inhibitors of trypsin-like serine proteases, e.g. tPA inhibitors. Inhibitors of interest include trypsin inhibitors, PMSF, APMSF, Antipain, Antithrombin, Leupeptin, 3,4-dichloroisocoumarin, TLCK, and the like, and those described in U.S. Pat. Nos.: 5,698,523; 5,693,617; 5,648,254; 5,580,561; 5,578,705; 5,567,602; 5,525,623; 5,523,390; 5,516,643; 5,506,114; 5,403,484; 5,367,064; 5,223,409; 5,153,176; 5,079,336; 4,963,654; 4,845,242; and 4,760,130, the disclosures of which are herein incorporated by reference. Preferred inhibitors are those which inhibit serine proteases known to be triggered by physiological stimulation, where a particularly preferred inhibitor is 4-(2-Aminoethyl)-benzenesulfonyl fluoride (i.e. AEBSF), described in Megyeri et al., Immunology (December 1995) 86:629-635, as well as mimetics thereof.

As mentioned above, an effective amount of the inhibitor is administered to the host, where “effective amount” means a dosage sufficient to produce a desired result. Generally, the desired result is at least a reduction in the protease-catalyzed cleavage of CAMs, where the magnitude of reduction is sufficient to result in treatment of the host. By treatment is meant at least an amelioration of the symptoms associated with the pathological condition afflicting the host, where amelioration is used in a broad sense to refer to at least a reduction in the magnitude of a parameter, e.g. symptom, associated with the pathological condition being treated, such as cell damage, intensity of seizure, seizure-proneness, brain damage and the like. As such, treatment also includes situations where the pathological condition, or at least symptoms associated therewith, are completely inhibited, e.g. prevented from happening, or stopped, e.g. terminated, such that the host no longer suffers from the pathological condition, or at least the symptoms that characterize the pathological condition.

In the subject methods, the protease inhibitor(s) may be administered to the host using any convenient means capable of resulting in the desired inhibition of proteolytic activity. Thus, the inhibitors can be incorporated into a variety of formulations for therapeutic administration. More particularly, the compounds of the present invention can be formulated into pharmaceutical compositions by combination with appropriate, pharmaceutically acceptable carriers or diluents, and may be formulated into preparations in solid, semi-solid, liquid or gaseous forms, such as tablets, capsules, powders, granules, ointments, solutions, suppositories, injections, inhalants and aerosols.

As such, administration of the compounds can be achieved in various ways, including oral, buccal, rectal, parenteral, intraperitoneal, intradermal, transdermal, intracheal,etc., administration. Preferably, the inhibitors will be sufficiently blood-brain permeable so that their administration into the systemic circulation will result in a therapeutically effective amount of inhibitor in the brain.

The inhibitors employed in the present invention can be administered alone, in combination with each other, or they can be used in combination with other known anti-seizure compounds (e.g. the antiepileptic compounds previously mentioned, GABA receptor agonists, GABA up-take inhibitors, etc.), as well as other protective compounds (e g., other protease inhibitors, calpain inhibitors, citicoline, anti-inflammatory agents, complement inhibitors, growth factors, glutamate receptor antagonists, antibodies, etc). With specific reference to stroke treatment, such serine protease inhibitors would likely also find use immediately after administration of exogenous tPA (which is used clinically to break-up blood vessel clots) to not only inhibit excess exogenous tPA activity but to also inhibit the cleavage of CAMs by endogenous tPA-like proteases. In pharmaceutical dosage forms, the inhibitors may be administered in the form of their pharmaceutically acceptable salts, or they may also be used alone or in appropriate association, as well as in combination, with other pharmaceutically active compounds. The following methods and excipients are merely exemplary and are in no way limiting.

For oral preparations, the compounds can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

The inhibitors can be formulated into preparations for injections by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

The compounds can be utilized in aerosol formulation to be administered via inhalation. The compounds of the present invention can be formulated into pressurized acceptable propellants such as dichlorodifluoromethane, propane, nitrogen and the like.

Furthermore, the compounds can be made into suppositories by mixing with a variety of bases such as emulsifying bases or water-soluble bases. The compounds of the present invention can be administered rectally via a suppository. The suppository can include vehicles such as cocoa butter, carbowaxes and polyethylene glycols, which melt at body temperature, yet are solidified at room temperature.

Unit dosage forms for oral or rectal administration such as syrups, elixirs, and suspensions may be provided wherein each dosage unit, for example, teaspoonful, tablespoonful, tablet or suppository, contains a predetermined amount of the composition containing one or more inhibitors. Similarly, unit dosage forms for injection or intravenous administration may comprise the inhibitor(s) in a composition as a solution in sterile water, normal saline or another pharmaceutically acceptable carrier.

The term “unit dosage form,” as used herein, refers to physically discrete units suitable as unitary dosages for human and animal subjects, each unit containing a predetermined quantity of compounds of the present invention calculated in an amount sufficient to produce the desired effect in association with a pharmaceutically acceptable diluent, carrier or vehicle. The specifications for the novel unit dosage forms of the present invention depend on the particular compound employed and the effect to be achieved, and the pharmacodynamics associated with each compound in the host.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

Preferred formulations of the inhibitors are oral preparations, particularly capsules or tablets containing each from about 10 milligrams up to about 1000 milligrams of active ingredient. The compounds are formulated in a variety of physiologically compatible matrixes or solvents suitable for ingestion or injection.

The inhibitors are administered at a dosage that at least reduces the amount of CAM cleavage that occurs in response to glutamate receptor stimulation as compared to a control while minimizing any side-effects. It is contemplated that the composition will be obtained and used under the guidance of a physician.

Typical dosages for systemic administration range from 0.1 to 50 milligrams per kg weight of subject per administration. A typical dosage may be 1-3 1000 mg tablets upon administration to an ER with seizure activity and/or stroke/headtrauma/hypoxia, or one 10-500 mg tablet taken once a day, or one time-release capsule or tablet taken once a day and containing a proportionally higher content of active ingredient. The time-release effect may be obtained by capsule materials that dissolve at different pH values, by capsules that release slowly by osmotic pressure, or by any other known means of controlled release.

Those of skill will readily appreciate that dose levels can vary as a function of the specific compound, the severity of the symptoms and the susceptibility of the subject to side effects. Some of the specific compounds that block proteolytic activity are more potent than others. Preferred dosages for a given compound are readily determinable by those of skill in the art by a variety of means. As mentioned above, a preferred inhibitor for use in the subject invention is AEBSF and mimetics thereof. With this preferred inhibitor, the dosage will generally range from about 0.1 to 50 mgs/kg, usually from about 1 to 30 and more usually from about 5 to 20 mgs/kg, depending, at least in part, on the condition being treated and the specific host.

A variety of hosts are treatable according to the subject methods. Generally such hosts are “mammals” or “mammalian,” where these terms are used broadly to describe organisms which are within the class mammalia, including the orders carnivore (e.g., dogs and cats), rodentia (e.g., mice, guinea pigs, and rats), and primates (e.g., humans, chimpanzees, and monkeys). In many embodiments, the hosts will be humans.

In addition to the subject methods, pharmaceutical compositions comprising unit dosages of the inhibitor sufficient to treat the pathological condition of interest are provided, where preferred compositions are capable of delivering the inhibitor to the extracellular synaptic regions of the brain. Furthermore, kits with unit doses of inhibitor, either in oral or injectable doses, are provided. In such kits, in addition to the containers containing the unit doses will be an informational package insert describing the use and attendant benefits of the drugs in treating pathological condition of interest. Preferred compounds and unit doses are those described herein above.

The following examples are offered primarily for purposes of illustration. It will be readily apparent to those skilled in the art that the formulations, dosages, methods of administration, and other parameters of this invention may be further modified or substituted in various ways without departing from the spirit and scope of the invention.

### Experimental

1. Detection of NMDA Receptor Mediated CAM Cleavage

A. Preparation of Tissue Cultures and Homogenates

1. Stimulation of Organotypic Cultures:

Standard methods were utilized for the preparation of cultured hippocampal slices from Sprague-Dawley rats at post-natal day twelve (Stoppini et al., “A simple method for organotypic cultures of nervous tissue,” J Neurosci. Meth. 37, 17382(1991)). The culture medium (Bahr, et al. “Stable maintenance of glutamate receptors and other synaptic components in long-term hippocampal slices,” Hippocampus 5, 425-439 (1995))(CM) was changed every other day. Slices were maintained in culture for 2-3 weeks and then subjected to a brief NMDA receptor stimulation paradigm (Thibault et al., “Long-lasting physiological effects of bath applied N-methyl-D-aspartate,” Brain Res. 476, 170-173 (1989) and Malenka, “Postsynaptic factors control the duration of synaptic enhancement in area CA 1 of the hippocampus,” Neuron 6, 53-60 (1991)). To activate NMDA receptors the slices were treated with 400 μM NMDA, 2 ; mM CaCl₂ (4 mM total) and 1 mM glycine. Control slices were also subjected to this cocktail but were first pretreated for 10 minutes with either 100 μM AP5 (an NMDA antagonist), or with kynurenic acid (2 mM) and MK-801 (100 μM), in CM. After 30 seconds of incubation with the NMDA cocktail, the solutions were removed and CM plus 2 mM AP5 was added to all slices. Tissue was harvested and processed for immunoblotting as described (See Bahr et al., supra). For analysis of soluble protein fractions, homogenized tissue was centrifuged at 330,000 g for 15 min. at 4° C. in a TL-100 rotor (Beckman Instruments). The resulting supernatant was extracted and treated for immunoblotting. Protein concentrations for the non-soluble pellets (i.e. all tissue left after the supernatant was extracted) were determined with a BSA standard curve. The protein value per pellet sample was used to calculate the amount of supernatant sample which corresponded to an equal starting amount of intact hippocampal tissue. Cleavage products were detected by immunoblotting with polyclonal rabbit antibodies (rabbit R7), (gift of B. A. Murray see [Murray & Jensen, “Evidence for heterophilic adhesion of embryonic retinal cells and neuroblastoma cells to substratum-adsorbed NCAM,” J. Cell Biol. 117, 1311-1320 (1992)]) which had been raised by multiple subcutaneous injections of affinity-purified NCAM prepared from detergent solubilized neonatal C57BL/6 mouse brain membranes by affinity chromatography using the 5B8 monoclonal antibody (Dodd et al., “Spatial regulation of axonal glycoprotein expression on subsets of embryonic spinal neurons,” Neuron: 1, 105-116 (1988)). Other antibodies used to label CAM fragments included anti-Ng-CAM from M. Grumet, and the “8342” antibody from E. Bock. Quantification of immunobands was determined without knowledge of sample type by densitometry.

2. Incubation of Hippocampal Homogenates:

Hippocampi were rapidly extracted from adult male Sprague-Dawley rats and submerged in ice-cold buffer (10 mM Tris-base, 100 mM NaCl; pH 7.4) homogenized on ice for 1 min in 5 mls of the same buffer and then centrifuged for 3 min. at 1000 g (4° C.). The pellet was discarded and the resulting supernatant was immediately incubated at 37° C. with or without EDTA, EGTA, or AEBSF. After incubation, samples were ultracentrifuged (as above) and processed for immunoblotting. Quantification of immunoblots was performed by densitometry with normalization of the immunoreactivity levels at 0 minutes to 100 percent (n=4 or 5; mean±S.E.M. for all time points).

B. Detection of 75 kDa Polypeptide in Tissue Cultures and Homogenates Western blot analyses revealed that brief applications of NMDA to cultured hippocampal slices prepared as describe above cause a marked increase in the concentration of a 75 kDa polypeptide recognized by polyclonal antibodies raised against NCAM. Specifically, the amount of the 75 kDa antigen (“CAM75”) in homogenates prepared from slices after a 30 second infusion of NMDA was 77±41% (mean±sd) greater than in slices exposed to NMDA and an antagonistic drug (p<0.001, n=9; two-tailed paired t-test). A 65 kDa species also present in the slices was unaffected by the NMDA treatment. Stimulating non-NMDA glutamate receptors (while NMDA receptors were blocked) had no effect on the concentration of CAM75 and nimodipine (20 μM), a blocker of voltage-gated calcium channels, did not block the increases elicited by NMDA. From these results, it is concluded that the rapid proteolysis is selective to NMDA receptors.

CAM75 is significantly smaller than intact NCAM isoforms but larger than their cytoplasmic domains, indicating that it incorporates a sizeable portion of the extracellular segment of the parent molecule. In agreement with this, an antibody (“8342”) raised against the extracellular fibronectin type III repeat region of NCAM labeled and immunoprecipitated CAM75. Also pointing to an extracellular origin, the cleavage product was present in soluble (high speed supernatant) fractions from the slices. Samples collected 30 seconds after a 30 second infusion of NMDA contained approximately twice the concentration of CAM75 as samples from control tissue (n=12, p=0.0003). Similar results were obtained with a 15 min delay after stimulation (n=4, p<0.05).

Given that the two antibodies which label CAM75 were raised against purified NCAM or a synthetic NCAM sequence, cross reactivity with anything other than another member of the highly homologous cell adhesion molecule family is very unlikely. The parent molecule is not NCAM itself because CAM75 was present in brain homogenates prepared from a mouse knockout (gift of H. Cremer, see Cremer et al. “Inactivation of the N-CAM gene in mice results in size reduction of the olfactory bulb and deficits in spatial learning. Nature, 1994: 367:455-9).

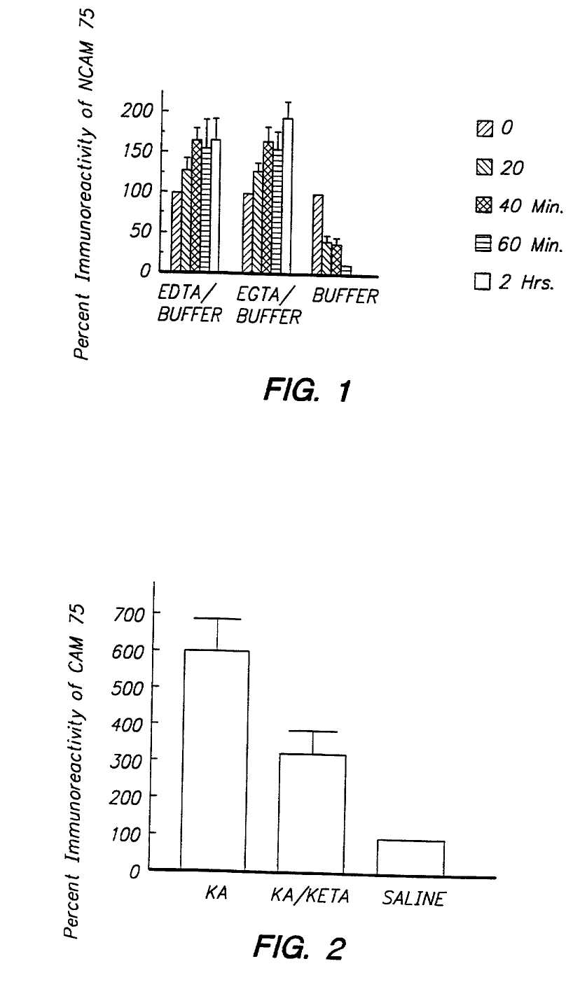

It has been previously demonstrated that a 65 kDa fragment is released from larger NCAM isoforms in a time-dependent manner. Similar results are obtained with brain homogenates, where a 65 kDa fragment and CAM75 are produced upon incubation in the presence of EDTA or EGTA (see FIG. 1). As noted, fragments similar in size to CAM75 are generated by cleavage of any of several NCAM relatives by serine-type proteases. That CAM75 also arises from an enzyme of this type was confirmed with the selective inhibitor 4-(2-Aminoethyl)-benzenesulfonyl fluoride (AEBSF); a 12±8% increase in the fragment occurred in membranes incubated for one hour with 500 μM of the inhibitor while a 45±8% increase was obtained in controls (n=3-5; p=0.03). Interestingly, when hippocampal homogenates were incubated in the absence of chelators CAM75 was rapidly degraded while concentrations of its 65 kDa counterpart steadily increased with time (FIG. 1). In contrast, CAM75 levels in soluble fractions without chelators did not change with time. Combining these results, CAM75 appears to be generated by an extracellular serine protease and partially digested by a membrane bound metalloprotease.

C. In vivo Detection of NMDA Receptor Mediated CAM Cleavage

Adult male rats were injected systemically with either: 10 mg/kg kainate (pH 7.4), 10 mg/kg kainate and 60 mg/kg ketamine (plus a second injection of 30 mg/kg ketamine one hour later), or saline. Behavioral responses were monitored (all animals in both sets injected with kainate reached seizure stages 4 or 5) and after two hours rats were anaesthetized, their hippocampi rapidly extracted and submerged in ice-cold buffer (10 mM Tris-base, 100 mM NaCl, 2 mM EDTA, and 2 mM EGTA; pH 7.4), homogenized on ice for 1 min in 5 mls of the same buffer, and then centrifuged for 3 min. at 1000 g (4° C.). The pellet was discarded and fractions of the resulting supernatants were immediately ultracentrifuged. Protein normalization and immunoblotting techniques were completed as described above. Quantification of CAM75 and the Ng-CAM fragment involved normalization of the immunoreactivity levels observed in saline injected rats to 100 percent (n=7-11; mean±S.E.M. for all groups).

From the above it was concluded that NMDA receptor driven proteolysis of cell adhesion molecules occurs in vivo.

FIG. 2 shows that CAM75 was increased by 598±85% in the hippocampus following approximately 45 minutes of periodic seizures resulting from a single injection of kainic acid (n=11, p=0.0002). The centrally active NMDA receptor channel blocker ketamine substantially reduced this effect (n=7, p=0.03; FIG. 2). Evidence for seizure induced extracellular cleavage at the consensus serine protease site was also obtained for a second adhesion molecule. Specifically, concentrations of an 80 kDa polypeptide detected by antibodies against Ng-CAM were increased by 603±140% (n=4, p=0.023) in ultrasupematant fractions from kainate injected rats.

From the above it was concluded that NMDA receptor activity results in the stimulation of extracellular proteolysis of cell adhesion molecules.

II. Identification of the Role of Trypsin-Like Serine Proteases in NMDA Mediated CAM Cleavage

A. Brain Serine Proteases Recognize a CAM Extracellular Serine Protease Site

Hippocampi were rapidly extracted from adult male Sprague-Dawley rats and submerged in ice-cold artificial cerebrospinal fluid (aCSF) which consisted of (in mM): 124 NaCl, 3 KCl, 1.25 KH₂PO₄, 2.5 MgSO₄, 3.4 CaCl₂, 26 NaHCO₃ and 10 glucose (pH 7.4), homogenized on ice for 1 min in 3 mls of the same buffer and then centrifuged for 5 min. at 1000 g (3° C.). The pellet was discarded and the resulting supernatant was ultracentrifuged at 200,000 g for 20 min. at 3° C. in a TLS-55 rotor (Beckman Instruments). The resulting ultrasupematant fraction was devoid of membrane-bound proteins and therefore represents a soluble protein fraction. This fraction was diluted to 50 μgs/ml in aCSF and incubated at room temperature with 100 μM of arginine linked to a fluorescent amido methyl-coumarin (AMC) substrate (Sigma Chemical Co), or to arginine-lysine-serine-arginine-AMC (Enzyme Systems Products; Livermore Cailf.) in the presence of differing concentrations of the serine protease inhibitor AEBSF (4-(2-Aminoethyl)benzenesulfonyl fluoride) (Sigma Chemical Co). Fluorescence was measured in a fluorometer (Sequoia-Turner Corp.) at 430 nm with stimulation at 360 nm. Quantification of proteolytic inhibition was performed by normalization of fluorescence in control tubes (i.e. those without added AEBSF) to 0 percent.

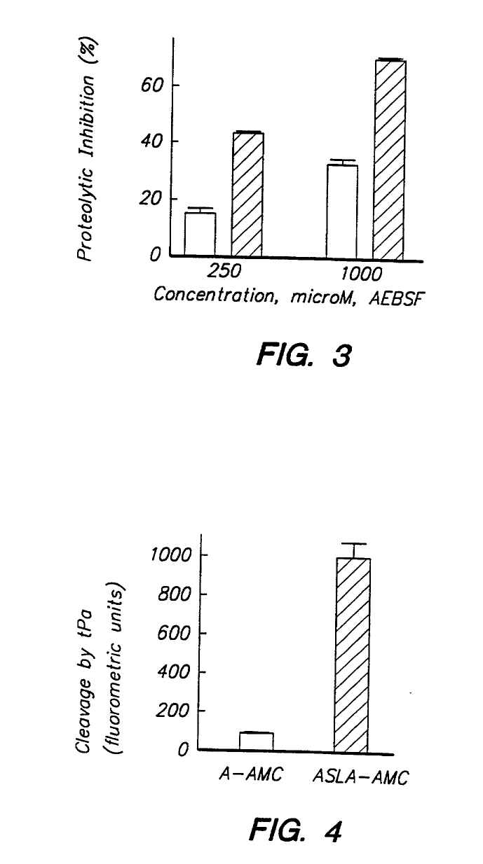

The serine protease substrate arginine-AMC (A-AMC) and a substrate made up of the CAM serine protease target sequence (arginine-serine-lysine-arginine) (ASLA-AMC) were both cleaved when incubated with soluble protein fractions (i.e. ultrasupernatants) from the hippocampus. This assay was then used to identify drugs and dosages that prevent extracellular CAM cleavage by endogenous proteases. The serine protease inhibitor AEBSF (4-(2-Aminoethyl)-benzenesulfonyl fluoride) inhibited proteolysis of the CAM sequence (ASLA-AMC) and did so to a much greater degree than it blocked the proteolysis of A-AMC. At 250 and 1000 uM A-AMC cleavage was blocked by 15 and 33 percent, respectively, while ASLA-AMC proteolysis was blocked by 44 and 70 percent. FIG. 3 (p≦0.0001).

The above results confirm that the brain contains proteases which recognize an extracellular consensus serine protease site located within the extracellular region of CAMs.

B. Incubation of Synthetic Substrates with tPA

1.5 μg/ml of tissue plasminogen activator (tPA, two chain, Sigma) was incubated at 23 ° C. with 40 μM ASLA- and A-AMC substrates (with or without the inhibitor AEBSF) in a final volume of 1.3 mls aCSF. After one hour of incubation the samples were put in the fluorometer and quantification of proteolytic activity normalized the readings from the A-AMC substrate to 100 (arbitrary units).

As shown in FIG. 4, tPA had a 10 fold preference for cleavage of the CAM substrate (ASLA-AMC) over the arginine-AMC substrate (n=6 for each substrate, p=0.002). The proteolytic activity of tPA was potently blocked by AEBSF at the same concentrations found in the brain ultrasupernatant experiments (not shown).

C. tPA Cleaves In Situ CAMs

That tPA cleaves in situ CAMs at the pertinent site was confirmed. tPA (6 μg/ml) was mixed with brain membranes [prepared as described in Hall et al, J. Neurochem. 59, 1997-2004 (1992)], for one, five, or twenty minutes (results indistinguishable so combined for quantification) at 23° C. Proteolysis was terminated by addition of 1 mM AEBSF and boiling in SDS-PAGE sample buffer. Addition of tPA to brain membranes caused a reliable increase (43±13%, mean±sd n=14, p=0.005) in a soluble CAM fragment of the same size and antigenicity as that induced by brief NMDA receptor stimulation, i.e. “CAM75”.

D. Consequences of AEBSF on Long-Term Potentiation

Brains from male Sprague-Dawley rats (190-225 g) were rapidly removed and placed into ice cold aCSF. Hippocampi were then cut transversely into 400-μm slices and transferred to a recording chamber perfused with aCSF (35° C.), where they were exposed to a humidified gas mixture of 95% oxygen and 5% carbon dioxide. After an hour recovery period, field recordings were made with a glass recording electrode containing 2 M NaCl positioned in the stratum radiatum of CAlb. NAC (Eclectex Enterprises; Irvine Cailf.) was used to record field EPSPs elicited by delivering single stimulation pulses (0.1 ms, 1-15 μA) to one of two bipolar electrodes positioned in Schaffer-commissural projections in CAla and CAlc. Each projection was stimulated every 20 sec with 10 sec interval between each electrode. After establishing a stable baseline (10-20 min), LTP was induced using a theta burst stimulation (TBS) paradigm [J. Larson et al., Brain Res. 368, 347-350 (1986)] in one pathway. After 30 minutes the perfusion media was switched to one containing 250 μM AEBSF. Forty-five minutes later TBS was applied to the non-potentiated pathway. The AEBSF/aCSF mixture was perfused for an additional 10 minutes, after which regular aCSF was added. In another set of slices TBS stimulation was applied during aCSF perfusion, and five minutes after LTP induction 250 μM AEBSF was infused for the remainder of the experiment. Potentiation for all groups was tabulated as the percent increase above initial baseline slopes.

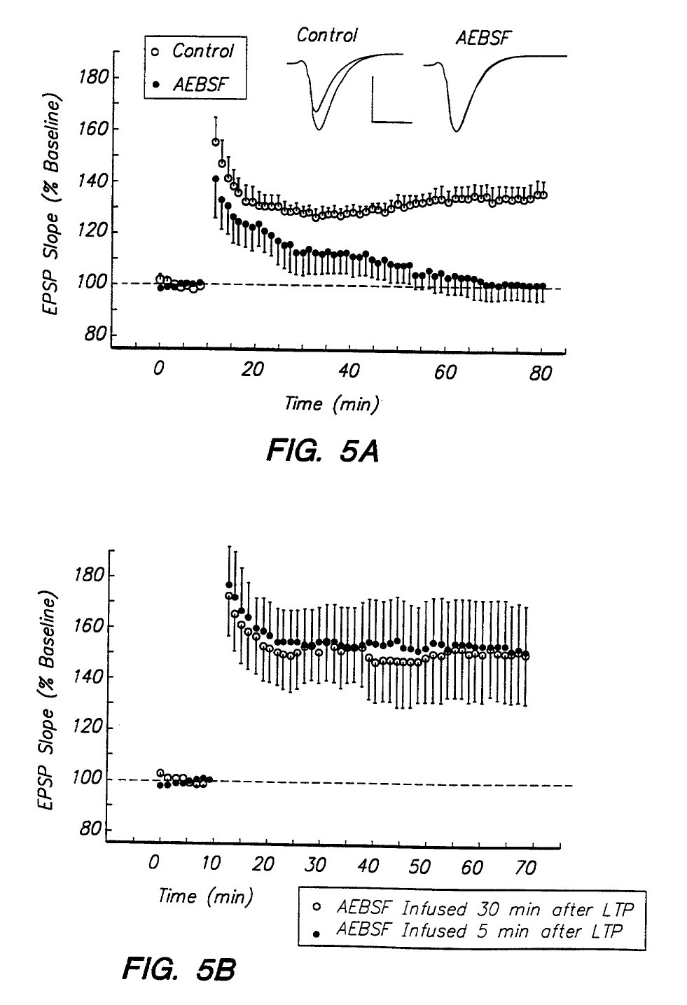

Infusion of 250 μM AEBSF for forty-five minutes had no significant effect on the slope, amplitude, or waveform of monosynaptic field EPSPs elicited within the CAl region by stimulation of the Schaffer-commissural projections. Application of theta burst stimulation at the end of this period resulted in an immediate and pronounced increase in the size of the synaptic responses. The magnitude of this effect varied considerably across slices but within slice differences between potentiation elicited before (control) vs. after (experimental) infusion of the drug could not be detected. However, the development of LTP in the minutes following induction was clearly different as is evident in FIG. 5A. Potentiation at control loci declined slightly for about ten minutes and did not decrease for the remaining 80 min of recording. In contrast, LTP induced after treatment with AEBSF decreased steadily until near baseline (pre-LTP) values were reached at about one hour post-induction. Mean percent potentiation at this time point was 103±5% for experimental and 134±3% for control pathways (n=6, p=0.005).

The serine protease inhibitor did not disturb already established LTP. As shown in FIG. 5B, infusions at five or thirty minutes after induction had no effects on potentiation over the subsequent hour. This accords with the rapidity (<1 min) with which extracellular proteolysis of CAMs occurs after activation of NMDA receptors. The lack of any drug related changes in response size in FIG. 5B also confirms the selectivity of AEBSF.

The above results demonstrate that NMDA receptor activation results in the release of a trypsin-like serine protease, e.g. tPA or relative thereof, into the extracellular synaptic space which results in the proteolysis of neuronal CAMs that, in turn, result in lasting synaptic changes, e.g. long-term potentiation.

III. AEBSF Inhibits Increases in Seizure-Proneness

Rats were divided into two groups: Group I rats were injected with 50 mgs/kg AEBSF and Group II (control) rats were injected with saline. The rats of both groups were then stimulated by direct electrical impulses to the dentate gyrus region of the hippocampus (40 times/5 mins apart)(a paradigm known to promote “kindling”, which can be thought of as an increase in seizure-proneness). The after-discharge duration (AD-duration) was then measured in each group. After a 10 day waiting period, the rats were again stimulated (5 times/20 mins apart)(no drugs were administered). The AD-duration was again measured. The results are provided in FIGS. 6 and 7.

The results demonstrate that the control rats showed ˜400% of the AD-duration upon the second testing period while rats injected once with AEBSF immediately prior to the first stimulation period showed no increase in AD-duration during the second testing period. These findings suggest that the inhibition of serine protease activity blocked the mechanisms by which seizures originate.

IV. AEBSF Inhibits Increases in Pre-Existing Seizure Proneness

The control group rats from III, above, were restimulated, with four of the rats of this group receiving a saline injection prior to stimulation and the remaining four rats receiving AEBSF (50 mgs/ml) injection prior to stimulations. Rats in the saline injected group showed enhanced AD duration when tested 10 days after stimulation, while the AEBSF injected group did not. The AD duration average of the control group was 207±103% of their original average, while the AD duration of the AEBSF injected rats was 26±77% of their original average. Two out of the four rats in the AEBSF injected group had no after discharges, i.e. no seizure-like activity.

From the above results it can be concluded that inhibition of serine proteases may be a viable treatment for pre-existing seizure states, such as chronic epilepsy. Since two out of four rats in the AEBSF group showed a complete loss of after-discharge activity, it can be concluded from the above results that such proteolytic inhibition may also reverse chronic epilepsy.

It is apparent from the above results and discussion that improved methods of treating pathological conditions associated with pathophysiology and excessive glutamate receptor stimulation are provided, where such conditions include those characterized by the presence of seizures, e.g. epilepsy, and neuronal demise, such as results from glutamate receptor over-activity resulting from hypoxia, head trauma and stroke. Because the methods focus on at least reducing one consequence of excitatory activity as seen in pathophysiology and glutamate-induced pathologies, i.e. CAM proteolysis, instead of merely treating symptoms resulting therefrom, they provide for improved results over currently employed methods of treating such pathological conditions. Furthermore, non-toxic active agents can be employed in the subject methods, thereby escaping toxic side effects associate with some current methods of treating neuronal demise.

All publications and patent applications cited in this specification are herein incorporated by reference as if each individual publication or patent application were specifically and individually indicated to be incorporated by reference. The citation of any publication is for its disclosure prior to the filing date and should not be construed as an admission that the present invention is not entitled to antedate such publication by virtue of prior invention.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

What is claimed is:

1. A method of inhibiting cell adhesion molecule cleavage in brain tissue of a host, said method comprising:
administering to said host an effective amount of a protease inhibitor.

2. The method according to claim 1, wherein said protease inhibitor is a serine protease inhibitor.

3. The method according to claim 1, wherein cell adhesion molecule cleavage in said brain tissue is associated with pathophysiology in said host.

4. The method according to claim 1, wherein cell adhesion molecule cleavage in said brain tissue is associated with neuropathology in said host.

5. A method for treating a host for a pathological condition associated with cleavage of cell adhesion molecules in brain tissue, said method comprising:
administering to said host an effective amount of a protease inhibitor.

6. The method according to claim 5, wherein said brain tissue is hippocampal tissue.

7. The method according to claim 5, wherein said cell adhesion molecules are present on the surface of neurons.

8. The method according to claim 5, wherein said cleavage is extracellular.

9. The method according to claim 5, wherein said protease is a serine protease.

10. The method according to claim 9, wherein said cell adhesion molecule has an extracellular domain comprising the residue sequence A-S-L-A and close relatives thereof.

11. The method according to claim 10, wherein said host is a mammalian host.

12. The method according to claim 5, wherein said host is suffering from a seizure, stroke, cerebral trauma or cerebral ischemia.

13. A method for treating a mammalian host for a pathological condition resulting at least in part from proteolysis of the extracellular domains of cell adhesion molecules, said method comprising:
administering to said host an effective amount of a serine protease inhibitor.

14. The method according to claim 13, wherein said proteolysis results from excessive glutamate receptor activity.

15. The method according to claim 13, wherein said glutamate receptor is an NMDA-type glutamate receptor.

16. The method according to claim 14, wherein said proteolysis results in neuronal demise.

17. The method according to claim 14, wherein said excessive glutamate receptor activity results from an event selected from the group consisting of stroke, head trauma and hypoxia.

18. The method according to claim 17, wherein said proteolysis results in long-term potentiation.

19. The method according to claim 17, wherein said proteolysis results in gains in excitatory responsiveness.

20. The method according to claim 18, wherein said pathological condition is characterized by the presence of seizures.

21. The method according to claim 20, wherein said pathological condition is epilepsy.

22. The method according to claim 13, wherein said serine protease is a trypsin-like-serine protease.

23. The method according to claim 22, wherein said inhibitor is a trypsin-like-serine protease inhibitor.

24. The method according to claim 23, wherein said inhibitor is a tPA inhibitor.

25. The method according to claim 24, wherein said inhibitor is AEBSF, and mimetics thereof.
